(19) United States
(12) Patent Application Publication
(10) Pub. No.: US 2007/0053899 A1
(43) Pub. Date: Mar. 8, 2007

(54) Activated carbon and use thereof for reducing or maintaining body weight

(75) Inventor: Todd J. McFarland, Cincinnati, OH (US)

Correspondence Address: WOOD, HERRON & EVANS, LLP, 2700 CAREW TOWER, 441 VINE STREET, CINCINNATI, OH, 45202, US

(73) Assignee: Senscina LLC

(21) Appl. No.: 11/219,224

(22) Filed: Sep. 2, 2005

(51) Int. Cl. A61K 33/44

(52) U.S. Cl. 424/125

(57) ABSTRACT

The present invention is generally directed towards compositions including activated carbon and methods of use thereof such as for reducing or maintaining body weight in a subject. In one embodiment, a dietary supplement comprising a composition including amorphous activated porous carbon particles, with a total surface area not less than about 700 m² per g and interstitial mesopores having a mean radius of not less than about 2 nm and not greater than about 50 nm, is administered to a subject in an amount sufficient, which may include from about 50 mg to about 50 g of activated carbon per dose, and for a duration sufficient, to reduce dietary uptake in the subject to reduce or maintain the body weight of the subject.

### FIELD OF THE INVENTION

The present invention pertains generally to activated carbon and to methods of use thereof, such as for reducing or maintaining body weight in a subject.

### BACKGROUND OF THE INVENTION

The increase in the numbers of overweight and obese adults and children in the United States has become alarming. It is estimated that Americans spend between 30 and 45 billion U.S. dollars on weight loss products, services, and programs every year. In addition, approximately 45 million Americans diet each year. Despite these statistics, the National Center for Health Statistics of the Center for Disease Control still says that about 64% of Americans over the age of 20 years are clinically overweight or obese and that 15% of children and adolescents ages 6 to 19 are overweight.

In the present context, the term “overweight” is used as an indication of a body with a weight exceeding the desirable weight, whereas the term “obesity” is used when the body weight is 20% or more above the desirable weight. Desirable weights for humans are given by the Council on Scientific Affairs defining the desirable weights for humans according to Metropolitan Height and Weight Tables as the midpoint of the range of the medium-frame individuals.

For a human subject to maintain a stable weight, an even balance between energy intake from food and energy expenditure should be maintained. Energy expenditure may be categorized in three basic ways: 1) energy expended during physical activity, 2) energy expended during rest, and 3) energy used to metabolize food.

When a person's caloric intake exceeds the amount of energy expended, the human body stores extra energy in the form of triacylglycerols in fat cells called lipocytes, which are present in adipose tissue. These lipocytes act as energy storage warehouses that can expand or contract as required. If the subject does not balance incoming and outgoing energy transfers by maintaining a sensible diet and a regular exercise regimen, the lipocytes engorge, causing fat build up. Over time, a person can gain weight becoming overweight, which may eventually lead to obesity.

Currently, there are a multitude of food, drug, and dietary supplements and other products, including lipase inhibitors, metabolism-boosting pills, herbal weight-loss teas, diet patches, and even jewelry that are being studied and/or marketed for weight loss and the treatment of obesity. Some items, such as soluble dietary fibers, are generally designed to block absorption of fats or carbohydrates. However, the safety and/or efficacy of various absorption blockers, e.g. chitosan, are either unknown and/or remain unsubstantiated.

If internal absorption blockade of fats in subjects, such as for the purpose of losing or maintaining body weight, is to be achieved in a manner safe enough for commercial use, at least several factors should be considered. First, the medium for absorption blockade should be capable of fat adsorption and should itself be unabsorbable in the subject, either completely or substantially, both alone and in an adsorbate form. The medium should be safe and administered in such a way so as to be convenient for the subject and in quantities sufficient to achieve the desired result. In addition, the medium should be of a highly lipophilic (non-polar) nature and consistently producible with Good Manufacturing Practices.

One known adsorptive medium that is not absorbable in subjects when ingested is activated carbon, which involves a carbon-based material typically with a high internal porosity and, hence, a large internal surface area. The primary raw material used for activated carbon may be any organic material having a high carbon content, e.g. coal, wood, peat, coconut shells, etc. Carbon may be extracted from these materials through various known processes. Three different pore types may be present in the activated carbon, including micropores (less than 2 nm in size), mesopores (2 to 50 nm in size), and macropores (greater than 50 nm in size). The method of processing activated carbon and/or use of different raw materials can provide activated carbons with different activities or physical properties.

The adsorptive properties of activated carbons have allowed for numerous applications spanning a wide range of industries. For example, activated carbon may be administered to humans to adsorb harmful drugs or toxic chemicals, such as to prevent death from an overdose. Activated carbon is also known to be used, for example, to treat diarrhea, flatulence, other miscellaneous intestinal disorders, hyperlipidemia, and liver, biliary tract, and renal diseases, and is used for beverage decolorization, sweetener purification, wastewater treatment, aquarium filters, swimming pool filters, gas masks, air filtration, and removal of pesticides.

Although the applications of activated carbons are numerous and span a wide range of industries, there is understood to be no known use of an activated carbon for reducing or maintaining body weight in people, such as for those individuals who are overweight or obese.

Thus, there is a need for a composition including activated carbon that is administered to subjects to reduce or maintain the body weight thereof.

### SUMMARY OF THE INVENTION

The present invention is directed generally towards a composition including activated carbon for reducing or maintaining body weight in human or animal subjects.

In one embodiment, the activated carbon includes amorphous activated porous carbon particles, which include a total surface area not less than about 700 m² per g and interstitial pores having a mean radius of not less than about 2 nm and not greater than about 50 nm. In one embodiment, the mesopores may be present in an amount of not less than about 5% of the total surface area of the carbon particles.

The activated carbon may be administered to a subject, such as before meals or snacks, in an amount sufficient and for a duration sufficient to reduce the lipophilic dietary uptake in the subject, via sorption, to reduce or maintain the body weight of the subject. In one embodiment, the amount sufficient includes no less than about 50 mg and no greater than about 50 g per dose. In another embodiment, the amount sufficient includes no less than about 400 mg and no greater than about 1200 mg of amorphous activated porous carbon particles per dose. The duration sufficient may include administration over the course of no less than about 1 day or no less than about 1 week. In another embodiment, the duration sufficient includes administration over the course of no less than about 4 weeks.

The activated carbon's adsorptive properties, affinity for adsorption of lipophilic substances, and its inability and its adsorbates' inability to be substantially absorbed through the human gut make it suitable for use as a dietary supplement for reducing or maintaining the body weight of a subject, such as an overweight or obese subject. In one embodiment, a dietary supplement, which may be in capsule form, includes the activated carbon in an amount, for example, no less than about 50 mg and no greater than about 600 mg per capsule. The dietary supplement may be ingested before a meal or snack by a subject in an amount sufficient to reduce caloric intake in the subject, such as by reducing lipophilic dietary unptake, and for a duration sufficient to reduce or maintain the body weight of the subject. In one embodiment, vitamins and/or minerals also may be provided in the supplement.

By virtue of the foregoing, there is thus provided a composition including activated carbon that is administered, e.g. in capsule form as a dietary supplement, to a subject in an amount and for a duration sufficient to reduce or maintain the body weight of the subject.

The features and embodiments of the present invention will become more readily apparent from the following Detailed Description.

### DETAILED DESCRIPTION OF THE INVENTION

The present invention is directed to a composition including activated carbon and to methods of use thereof such as for reducing or maintaining body weight in human or animal subjects. As further discussed below, the activated carbon may be used to reduce the absorption of lipids and/or carbohydrates, such as fats, cholesterol, sugars, starches, or other unwanted substances in the diet of human and animal subjects. In this way, caloric intake is reduced so that a subject may reduce or maintain body weight.

In accordance with the present invention, the activated carbon includes amorphous activated porous carbon particles. In one embodiment, these porous carbon particles include a total surface area no less than about 700 m² per g and interstitial pores having a mean radius of no less than about 2 nm and no greater than about 50 nm. Pores of no less than about 2 nm and no greater than about 50 nm are considered mesopores with micropores being less than 2 nm in size and macropores being greater than 50 nm in size. The number of interstitial mesopores, i.e. pores having a mean radius of no less than about 2 nm and no greater than about 50 nm, is maximized according to the desired surface area, i.e. no less than about 700 m² per g. In one embodiment, the mesopores are present in an amount of not less than about 5% of the total surface area of the carbon particles. In another embodiment, the mesopores are present in an amount of not less than about 8% of the total surface area of the carbon particles. In yet another embodiment, the mesopores are present in an amount of not less than about 15% of the total surface area of the carbon particles.

The combination of the surface area and number of mesopores, expressed as percent of the total surface area, is believed to provide the carbon particles with sufficient sorptive properties for targeting the desired compounds as well as intermediaries of the human diet, including lipids and/or carbohydrates, such as fats, fatty acids, triacylglycerols, polysaccharides, phospholipids, cholesterol, etc.

The activated carbon of the present invention may be prepared by any known method, but which typically involves a two-step process: 1) the carbonization of carbonaceous precursors (e.g. coal, wood, peat, bamboo, etc.) at higher temperatures to separate or extract the carbon from other materials and 2) the activation of the carbonized product. After processing, the activated carbon is understood to be organized in groups of aromatically arranged carbon atoms in irregularly cross-linked layers or sheets giving rise to carbon particles having interlayer spaces defining the porosity and surface area. The surface of the carbon particles is inundated with charged carbon-oxygen and carbon-hydrogen groups.

While not intending to be held to the specific details, the following is one proposed mechanism (or theory) by which the activated carbon of the present invention may be effective for weight loss. The activated carbon is able to reduce the absorption by the body of lipophilic substances, e.g. lipids such as fats, which have been ingested by a subject and emulsified by bile acids. More specifically, the non-polar hydrophobic nature of the activated carbon provides a physiochemical preference for adsorption onto the surface thereof of non-polar organic species including lipophilic substances, such as emulsified fats. After being adsorbed onto the particle's surface, the carbon particles, which are inundated with charged carbon-oxygen and carbon-hydrogen groups, catalyze the movement of the lipophilic substance into the mesopores by weak molecular van der Waals forces. Once the surface-adsorbed substance is absorbed into the interstitial mesopores of the carbon particle, the substance is essentially trapped. This is believed to be accomplished by both the physical barrier of the pore boundaries and the charge present on the surface of the particle.

Once trapped, the substance(s), along with the non-absorbable activated carbon, is moved through the body and eventually excreted. In this way, the activated carbon composition reduces the lipophilic dietary uptake in the subject resulting in reduced caloric intake, which allows the subject to reduce or maintain body weight. Other substances that may be sorbed by the carbon particles include other lipids and/or any carbohydrate such as cholesterol, chylomicrons, mono- and polysaccharides, glycerols, phospholipids, etc. In one embodiment, the activated carbon also may be used to lower or maintain cholesterol levels by reducing the absorption of cholesterol in subjects.

In one method of the present invention, the composition including the activated carbon is administered, e.g. orally administered, to a subject in an amount and for a duration sufficient to reduce the lipophilic dietary uptake in the subject to reduce or maintain the body weight of the subject. As indicated above, in one embodiment, the amorphous activated porous carbon particles include a total surface area not less than about 700 m² per g and interstitial pores including a mean radius of not less than about 2 nm and not greater than about 50 nm. The mesopores are present in an amount of not less than about 5% of the total surface area of the carbon particles.

The amount sufficient of activated carbon particles, in one embodiment, includes not less than about 50 mg and not greater than about 50 g of amorphous activated porous carbon particles per dose. In another embodiment, the amount sufficient includes not less than about 200 mg and not greater than about 1400 mg of amorphous activated porous carbon particles per dose. In yet another embodiment, the amount sufficient includes not less than about 400 mg and not greater than about 1200 mg of amorphous activated porous carbon particles per dose. In one embodiment, the dose should not exceed about 4 g per day.

The duration sufficient can include administering to the subject the composition over the course of about one day. In another embodiment, the duration sufficient is not less than about one day. In another embodiment, the duration sufficient is not less than about one week. In yet another embodiment, the duration sufficient is not less than about four weeks.

In one embodiment, the dose is administered before a meal or snack. A meal is defined as the ingestion of 700 or more calories with a snack being defined as the ingestion of less than 700 calories. In another embodiment, the dose is administered about 30 minutes before the meal or snack.

In another embodiment, the carbon particles include interstitial pores having a mean radius of not less than about 5 nm and not greater than about 20 nm, and the total surface area may be not less than about 900 m² per g. In yet another embodiment, the surface area is not less than about 1100 m² per g. In another embodiment, the surface area is not less than about 1100 and not greater than about 1200 m2 per g. In one embodiment, the purity of ash content of the activated carbon may be not less than about 10% by weight. In another embodiment, the ash content is not less than about 5% by weight.

The sorptive properties of the activated carbon of the present invention make it ideally suited for use as a dietary supplement for reducing or maintaining the body weight of a subject, such as an overweight or obese subject. Accordingly, in one example, a dietary supplement, which may be in capsule form, is administered to a subject in an amount and for a duration effective, as discussed above, to adsorb and absorb substances in the stomach and small intestine, rendering them unabsorbable through the gastrointestinal tract. In this way, the dietary supplement reduces lipophilic dietary uptake in the subject to reduce or maintain the body weight of the subject.

In one embodiment, a capsule includes the composition comprising activated carbon in an amount not less than about 50 mg and not greater than about 600 mg per capsule. In another embodiment, a capsule includes the composition comprising activated carbon in an amount not less than about 300 mg to about 500 mg. In yet another embodiment, a capsule includes about 400 mg. One to three capsules may be taken before meals or snacks, such as about 30 minutes before. In one embodiment, one to two capsules are taken before snacks and two to three capsules are taken before meals not to exceed about ten capsules per day.

A non-limiting example of the effects of using activated carbon for reducing or maintaining weight in a subject in accordance with the present invention is presented below.

### EXAMPLE

Fifteen subjects including five men and ten women (ranging in age from 26-65 years) were instructed to take up to 1 g of a commercially purchased activated carbon (Nature's Way Charcoal Activated Caps from Nature's Way of Springville, Utah for controlling flatulence) 30 minutes prior to meals, with meals being defined as the ingestion of 700 or more calories. Subjects also were instructed to take up to 0.5 g 30 minutes prior to snacks, with snacks being defined as the ingestion of less than 700 calories. Subjects were further instructed not to alter their normal diet and exercise regimen while participating in the study. Subjects were provided a 21-day supply.

Compliance rates varied greatly. None of the subjects administered the carbon a recommended 30 minutes prior to meals. Many administered the dose immediately prior to or with meals, and some administered the dose following meals. Approximately ⅓ of the subjects described themselves highly compliant in remembering to administer the activated carbon before every meal.

The average weight loss for the group was 5.66 lbs over 3.87 weeks for a resulting average of 1.47 lbs per week. Three subjects who continued usage for eight weeks reported weight loss ranging from 10-16 lbs. The majority of subjects averaged a weight loss of approximately 2 lbs per week. Four subjects reported no weight loss. One discontinued after only one week, citing no weight loss. Two discontinued after two weeks of use, citing no weight loss, but acknowledging poor compliance.

Results

| | | Average |
|---|---|---|
| Number of subjects: | 15 | |
| Time Period (weeks): | 1-8 wks | 3.87 wks |
| Age: | 26-65 yrs | 38.2 yrs. |
| Sex: | 5 Male; 10 Female | |
| Weight Loss (lbs): | 0-16 lbs. | 5.66 lbs. |
| Dose (g/day): | 0-4 g/day | |
| Compliance Rate: | Variable | |

Average weight loss in one week: 1.47 lbs
Average weight loss per month: 5.88 lbs

Based on the resulting average weight loss among participants, the total caloric intake among subjects was reduced by an average of 700 calories per day, which equates to a weekly caloric reduction of approximately 5,000 calories or 1.42 lbs of body weight. A reduction of 700 calories is equivalent to an approximate 31.8% reduction in calorie intake based on a daily average consumption of 2,200 calories.

Concerning administration of the activated carbon, one skilled in the art will appreciate that many suitable methods are available, and, although more than one route may be used, a particular route of administration may be more effective. As such, the composition including the activated carbon according to the present invention may be formulated for administration by any suitable enteral or parenteral route, as known to one skilled in the art. Therefore, the composition may be in the form of tablets such as chewables, capsules such as gelatin capsules, suspensions, liquids, gum, emulsions, solutions, suppositories, and in other suitable forms, such as dragee, powder, sachet, pellet, granule, or non-pareil.

Formulations for oral use that may be presented as soft gelatin capsules may have the activated carbon composition mixed with water or an oil medium, for example, peanut oil, liquid paraffin, or olive oil as well as colorants, stabilizers, and other fillers. Powders, dispersible powders or granules suitable for preparation of an aqueous suspension by addition of water may also be convenient dosage forms of the present invention.

The activated carbon composition, as indicated above, may also be administered to animals (e.g. domestics and non-domestics) such as to improve the performance of the animal (e.g. daily weight gain and feed utilization) or to improve carcass quality or both. Carcass quality is generally improved when the fat tissue mass/lean mass body mass ratio is decreased, i.e., when the body content of meat is increased e.g., at the expense of the body content of fat.

Accordingly, there is thus provided a composition including activated carbon that is administered, e.g. in capsule form as a dietary supplement, to a subject in an amount and for a duration sufficient to reduce or maintain the body weight of the subject.

While the present invention has been illustrated by the description of the various embodiments thereof, and while the embodiments have been described in considerable detail, it is not intended to restrict or in any way limit the scope of the appended claims to such detail. Additional advantages and modifications will readily appear to those skilled in the art. The invention in its broader aspects is therefore not limited to the specific details, representative methods and examples shown and described. Accordingly, departures may be made from such details without departing from the scope of Applicant's general inventive concept.

What is claimed is:

1. A method for reducing dietary uptake in a subject, comprising:
administering to a subject a composition comprising activated carbon including amorphous activated porous carbon particles having a total surface area not less than about 700 m² per g and interstitial mesopores including a mean radius of not less than about 2 nm and not greater than about 50 nm in a dose and for a duration sufficient to reduce dietary uptake in the subject to reduce or maintain the body weight of the subject.

2. The method of claim 1, wherein the dose includes not less than about 50 mg and not greater than about 50 g of amorphous activated porous carbon particles per dose.

3. The method of claim 1, wherein the dose includes not less than about 400 mg and not greater than about 1200 mg of amorphous activated porous carbon particles per dose.

4. The method of claim 3, wherein the dose does not exceed about 4 g per day.

5. The method of claim 3, wherein the dose is administered before a meal or snack.

6. The method of claim 5, wherein the dose is administered about 30 minutes before the meal or snack.

7. The method of claim 1, wherein administering to the subject a composition for a duration sufficient includes administering to the subject the composition over the course of not less than about one day.

8. The method of claim 7, wherein administering to the subject a composition for a duration sufficient includes administering to the subject the composition over the course of not less than about one week.

9. The method of claim 8, wherein administering to the subject a composition for a duration sufficient includes administering to the subject the composition over the course of not less than about four weeks.

10. The method of claim 3, wherein the dose is provided in the form of a capsule.

11. The method of claim 1, wherein the interstitial mesopores have a mean radius of not less than about 5 nm and not greater than about 20 nm and the total surface area is not less than about 1100 m² per g, the mesopores being not less than about 5% of the total surface area of the carbon particles.

12. A method for reducing or maintaining the body weight of a subject, comprising:
administering to the subject a composition comprising activated carbon including amorphous activated porous carbon particles in a dose and for a duration sufficient for reducing or maintaining the body weight of a subject.

13. The method of claim 12, wherein the dose includes not less than about 50 mg and not greater than about 50 g of amorphous activated porous carbon particles per dose.

14. The method of claim 13, wherein the dose is administered before a meal or snack.

15. The method of claim 12, wherein administering to the subject a composition for a duration sufficient includes administering to the subject the composition over the course of not less than about one day.

16. The method of claim 15, wherein administering to the subject a composition for a duration sufficient includes administering to the subject the composition over the course of not less than about one week.

17. The method of claim 12, wherein the amorphous activated carbon particles include a total surface area of not less than about 700 m² per g and interstitial meospores having a mean radius of no less than about 2 nm and not greater than about 50 nm, the mesopores being not less than about 5% of the total surface area of the carbon particles.

18. The method of claim 17, wherein the surface area is not less than about 1100 m² per g and the interstitial mesopores have a mean radius of not less than about 5 nm and not greater than about 20 nm.

19. The method of claim 13, wherein the dose is provided in the form of a liquid or one or more pills, tablets, or capsules.

20. A method of making a dietary supplement, comprising:
obtaining a composition comprising activated carbon including amorphous activated porous carbon particles, the amorphous activated porous carbon particles having a total surface area not less than about 700 m² per g and interstitial mesopores including a mean radius of not less than about 5 nm and not greater than about 20 nm, the mesopores being not less than about 5% of the total surface area of the carbon particles; and
encapsulating the composition comprising activated carbon in an amount not less than about 50 mg and not greater than about 600 mg per capsule to result in an ingestible dietary supplement, the dietary supplement capable of reducing caloric intake in a subject to reduce or maintain the body weight of the subject when ingested.

21. A dietary supplement, comprising:
an ingestible capsule containing an activated carbon composition including amorphous activated porous carbon particles in an amount not less than about 50 mg and not greater than about 600 mg per capsule, the amorphous activated porous carbon particles having a total surface area not less than about 700 m² per g and interstitial mesopores including a mean radius of not less than about 5 nm and not greater than about 20 nm, the mesopores being not less than about 5% of the total surface area of the carbon particles, the dietary supplement capable of reducing dietary uptake in a subject to reduce or maintain the body weight of the subject.
